(19) United States
(12) Patent Application Publication
(10) Pub. No.: US 2024/0189382 A1
(43) Pub. Date: Jun. 13, 2024

(54) FORMULA FOR INHIBITING AGING REGENERATION REPAIR

(71) Applicant: Haohan Xu, Shenzhen (CN)

(72) Inventor: Haohan Xu, Shenzhen (CN)

(21) Appl. No.: 18/198,889

(22) Filed: May 18, 2023

Related U.S. Application Data
(63) Continuation-in-part of application No. PCT/CN2021/131068, filed on Nov. 17, 2021.

(30) Foreign Application Priority Data
Nov. 19, 2020 (CN) 202011299050.6

(51) Int. Cl. A61K 36/82; A61K 9/00; A61P 1/02; A61P 1/14; A61P 11/02; A61P 17/02; A61P 27/02; A61P 29/00

(52) U.S. Cl. CPC A61K 36/82; A61K 9/0014; A61K 9/0043; A61K 9/0048; A61K 9/0053; A61P 1/02; A61P 1/14; A61P 11/02; A61P 17/02; A61P 27/02; A61P 29/00

(57) ABSTRACT

Inflammation and aging slow down the recovery rate of the elderly, making it extremely difficult for them to restore their bodies to a youthful state. Age-related deterioration of bodily tissues and organ functions often manifest as inflammation and tumors, which are key factors in inhibiting aging. Researchers have found that the body's nervous system is closely related to initiating the body's regenerative repair functions. The formulation solution is actively absorbed by the human nervous system, achieving drugs delivery over long distances along the nervous system. The recovery speed of the elderly can be accelerated by using formula disclosed in this application. This application combines anti-aging and regeneration, emphasizing the increase in concentration of sodium ions and neurotransmitter-like small molecules in the nervous system as key factors for repair and regeneration. This application belongs to the field of biomedicine.

### CROSS-REFERENCE TO RELATED APPLICATIONS

The present application is a continuation-in-part application of PCT application No. PCT/CN2021/131068 filed on Nov. 17, 2021, which claims the benefit of Chinese Patent Application No. 202011299050.6 filed on Nov. 19, 2020. The contents of all of the aforementioned applications are incorporated by reference herein in their entirety.

### TECHNICAL FIELD

The present application belongs to the field of biomedicine and relates to a formula for the prevention or treatment of painful inflammatory disorders of the nervous system, inflammatory disorders of the immune system, and degenerative disorders of the nervous system, and in particular relates to the use of the formula and its derivatives in the preparation of drugs for the prevention and/or treatment of painful diseases, inflammatory diseases of the nervous system, and degenerative disorders of the nervous system.

Inflammation and aging cause the body of the elderly to return to health at a slower rate, and it is extremely difficult for the elderly to return to the physical state of youth; researchers have found that the body's nervous system and the initiation of the body's regenerative and restorative functions are closely related.

The formula of this application can accelerate the speed of recovery of the elderly. This application is a combination of inhibition of aging and regeneration. Due to age-related decline in the function of body tissues and organs, inflammation and tumors are common manifestation of traits, inhibition of inflammation and tumors is the key point of inhibition of aging. At the same time, the increase of the concentration of sodium ions and nutrients of small molecules, especially small molecules of neurological transmitters is the key point of repair and regeneration.

### BACKGROUND

Research shows that aging can be understood as a result of the accumulation of multiple chronic inflammatory diseases, specifically characterized by the long duration of inflammation, the escalation of inflammation into cancer in some populations, the decline of neurotransmitter levels, the decline of muscle mass, the degenerative diseases resulted in the decline of the immune system, such as the degenerative diseases of the eyes, brain, spine, reproduction, limb movement and other aspects. These characteristics pose significant challenges to the prevention and treatment of aging. Although there are many regenerative medicine such as stem cells, gene editing, and liposomal drug delivery modalities in the current medical technology, effective therapeutic drugs with low side-effects are still insufficient. Therefore, preventive methods and therapeutic measures to inhibit aging need to be developed urgently.

### SUMMARY

In order to solve the technical problem of inhibiting aging and preventing and treating diseases caused by aging, the present application provides a formula for inhibiting aging and promoting regeneration and repair, which enables the human nervous system to actively absorb the formulation solution. The human nervous system actively absorbs the formulation solution, which is then converted into multiple types of neurotransmitters, enhancing the level of the human body's electrical signals. Stronger levels of electrical signals can effectively inhibit inflammation, inhibit and control bacterial and viral infections, block the transmission of inflammatory pain signals, and non-invasively deliver the drug along a fixed pathway in the nervous system; thus realizing the long-distance drug administration and delivery in the human nervous system.

The present application also provides applications of the above formulations in the preparation of drugs for the prevention and/or treatment of degenerative disorders.

This application is realized through the following technical solution:

a formula for inhibiting aging and promoting regeneration and repair, which can bind with the human nervous system and administer medication over long distances along fixed pathways of the nervous system.

The present application is developed to inhibit degenerative disorders from the perspective of active absorption by the nervous system, which is capable of inhibiting the binding of inflammatory substances to healthy tissues at different stages of disease progression. The use of the nervous system to deliver drugs to the brain and spine has not yet been reported.

Specifically, the formulation is for inhibiting aging and promoting regeneration and repair. The formula comprises tea and sodium chloride. The tea consists of any combination of one or more types of tea, and the higher the concentration of the tea component in the formulation solution, the higher the concentration of sodium chloride, the better and more stable the implementation.

The types and proportions of amino acids contained in different teas are relatively similar.

Only one type of salt, sodium chloride, was chosen in the formula because of the stability and effectiveness of the experimental results. One of the formulas consists of a combined solution of Tieguanyin tea and sodium chloride, with sodium chloride reaching a saturated concentration (about 26.47%), at which point the solubility is about 36. Sodium chloride reaches a saturated concentration, which dramatically extends the half-life of small molecules such as amino acids, inducing a dramatic increase in bacterial viral membrane permeation, and increasing the effectiveness of inhibiting viruses and bacteria.

Cytological experiments show that: according to the literature of Prof. Wu Yonglin of Tianjin University, the use of tea has a better antibacterial mechanism against methicillin-resistant Staphylococcus aureus, and there is an excellent biosafety in swine experiments. The effectiveness of nebulized inhalation treatment in the treatment of a mouse model of lethal H1N1-MESA mixed pneumonia is attributed to the fact that the tea molecules accumulate on the surface of the bacteria, inducing a gradual permeation of the MRSA membrane and destroying the entire membrane structure thereby killing the bacteria.

According to news reports in 2020, the Zhejiang Provincial Center for Disease Control and Prevention selected two grams each of green tea, Tieguanyin, and Dahongpao. They were steeped in 50 milliliters of boiling water (measured at 90 degrees Celsius) for 30 minutes, resulting in a concentration of 40 mg/ml (typical for the initial brew of tea). The solution was then cooled to room temperature and diluted with cell culture medium to the desired experimental concentrations. In the experiment, different concentrations of tea solutions (2.5-10 mg/ml) were used for in vitro antiviral experiments. After the 100tcid50 novel coronavirus is treated with the tea with different concentrations and the control group, the Vero was infected and cultured for 48 hours. The level of viral nucleic acid proliferation was measured. The results showed that the nucleic acid proliferation of the novel coronavirus in the tea-treated group was reduced by 10,000 to 100,000-fold, and virus inactivation was close to 100%, when compared with the virus control group without tea treatment.

Through the study, the effect of tea polyphenols and sodium chloride is poor in the lung nebulization, skin anti-inflammatory, oral experiments. The test effect of the use Tieguanyin tea and sodium chloride is better. This is because the natural ingredients of tea are similar to neurotransmitter ingredients.

Through the study, other plants comprising nearly a hundred plants, such as Isatis roots, Astragalus membranaceus, Forsythia suspensa, Mulberry leaves, Ginger, Lycium barbarum, Chrysanthemum, Cassia seed, Poria cocos, and many others, and sodium chloride were used in the nebulization experiments conducted on the lungs. However, the results showed poor efficacy or significant side effects, such as inflammation and pain. On the other hand, the use of Tieguanyin tea combined with sodium chloride demonstrated stable effectiveness, safety, and no side effects.

A preparation method for the formula for inhibiting aging and promoting regeneration and repair is provided, and its involves synthesizing the formula through physical methods.

An application of formula for inhibiting aging and promoting regeneration and repair in the preparation of drugs for prevention and/or treatment for age-related degenerative diseases. The diseases include at least one disease selected from the following group: the inflammation of the nervous system, the inflammation of the immune system, pain caused by cancer and pain caused by inflammation, degenerative disorders of the nervous system and inflammation of other organs in the human body.

A drug for prevention and/or treatment of the inflammation of the nervous system, the inflammation of the immune system and the degenerative disorder of the nervous system, wherein, the dosage form of the drug is any one of a spray, an oral preparation, an injectable preparation, a tablet, a capsule, a granule, a suspension mixture and a pill.

One or more technical solutions in embodiments of the present application have at least the following technical effects or advantages:

Firstly, a formula for inhibiting aging and promoting regeneration and repair specifically contains tea and sodium chloride. which can be combined with the human nervous system. The formula solution is actively absorbed by the human nervous system, and then converted into multiple types of neurotransmitters to enhance the body's electrical signal level, and then is administered along the fixed path of the nervous system in the location of the eyes, nose, mouth, nipples, genitals, rectum, etc., and therefore is delivered to all the nervous system of the whole body, so as to realize the Long-distance drug delivery for the human nervous system. A stronger electrical signal level can effectively inhibit inflammation, inhibit and control bacterial and viral infections, and block the transmission of inflammatory pain signals.

Secondly, a drug for the prevention or treatment of aging and degenerative disorders, which can bind with the human nervous system, which can be actively absorbed by the human nervous system. The absorbed ingredients are delivered to vesicles. As the concentration of sodium ions and chloride ions outside the cell membrane of human nerve cells is significantly increased, the concentration of various amino acids, enzymes, vitamins and other ingredients is also significantly increased. Some of the ingredients of the formula is converted into neurotransmitters for use in signal transmission by the human nervous system. The concentration of sodium ions outside the human nerve cell membrane has increased from 150 mmol/l to 4524 mmol/l. This concentration of 4524 mmol/l is much higher than the concentration of 451 mmol/l found in malignant tumors and the concentration of 200-300 mmol/l found in inflammation. Therefore, the resulting electrical signal can hinder the transmission of pain signals from malignant tumors and inflammatory pain. Simultaneously, it inhibits the replication of bacteria and viruses and suppresses inflammation while blocking the transmission of pain signals. The concentration is increased from 150 mmol/l to 4524 mmol/l, then decreased from 4524 mmol/l back to 150 mmol/l; and increased again from 150 mmol/l to 4524 mmol/l. Plotting the concentration values of sodium ion against time on the horizontal axis will yield a spiral-shaped graph depicting the changes in the concentration of sodium ions. The concentration of sodium ions outside the nerve cell membrane of regenerative animals, such as octopuses, is 440 mmol/l. Therefore, it is speculated that each time the concentration of sodium ions outside the human nerve cell membrane exceeds 440 mmol/l, the signal for human regeneration and repair is triggered. By accumulating these signals in a regular manner, the inflammation and degenerative diseases is possible to be inhibited, thereby aging and pathological changes in human tissues and organs can be effectively suppressed.

Thirdly, a drug for the prevention or treatment of aging and degenerative diseases is provided, which can be administered noninvasively along a fixed pathway in the nervous system. The usage time and dosage are as follows: 1-2 times per day; for the eyes, the usage time is approximately one minute each time, and the dosage is 0.1-1 milliliters each time; for the nose, the usage time is approximately five minutes each time, and the dosage is 0.1-1 milliliters each time; for the mouth, the usage time is about 2 minutes each time, and the dosage is 5-10 milliliters each time; for the lung atomization, the usage time is about 30 minutes each time, and the dosage is 5-20 milliliters each time; for the reproductive organs, the usage time is about 10-20 minutes each time, and the dosage is 5-10 milliliters each time; for the nipples, the usage time is about 10 minutes each time, and the dosage is 10 milliliters each time; for the rectum, the usage time is about one minute each time, and the dosage is 5-10 milliliters each time; for the skin, the usage time is about 5-10 minutes each time, and the dosage is 60 milliliters each time. Regular administration can achieve a stable regenerative effect while replenishing neurotransmitter levels in elderly individuals.

Fourthly, the fixed paths for non-invasive drug administration include: the drug administrated to the eyes is delivered to the heart through the occipital bone and the sacrum; the drug administrated to the nose is delivered to the ear through the trigeminal nerve; the drug administrated by nasal and oral atomization is delivered to the lungs and cervical nerves; the drug administrated through the mouth is delivered to the dental nerves, the neck and the stomach; the drug administrated through the rectal administration is delivered to the stomach and the neck along the digestive system; the drug administrated through the rectal administration is delivered to the to the sacral plexus and the nerves surrounding the anus; the drug administrated through the external genitalia is delivered to the sacral plexus, lumbar plexus, sciatic nerve, plantar nerves, superior gluteal nerve, inferior gluteal nerve, sexual nerves, urinary bladder, kidneys, and so on; the drug administrated through the nipple is delivered to the thoracic dorsal nerve, brachial plexus, cervical nerves, ulnar nerve, hand, facial nerve; other methods of administration such as injection, with a minimal injection volume of approximately 0.1 milliliters each time.

### DETAILED DESCRIPTION OF THE PREFERRED EMBODIMENT

The following will combine specific implementation methods and examples to elaborate on this application, presenting the advantages and various effects of this application more clearly. Those skilled in this field should understand that these specific implementation methods and examples are used to illustrate the application, rather than limit it.

Throughout the specification, unless otherwise specified, the terms used in this application should be understood in the meaning commonly used in this field. Therefore, unless defined otherwise, all technical and scientific terms used in this application have the same meaning as generally understood by those skilled in the field described in this application. In case of conflicts, this specification shall prevail.

Unless otherwise specified, all kinds of raw materials, instruments and equipment used in this application can be purchased from the market or prepared by existing methods.

In 2012, suffered from years of headaches due to vascular abnormalities in the brain, which severely affected my ability to walk at times, the hospital examinations revealed lacunar infarction with cavities in both the left and right hemispheres of my brain. As a result, I could only engage in some light physical research and development work, such as observing the combustion of smoke and haze, and combining mist generated by plant sodium chloride to accelerate the settling of haze, effectively removing benzene and pyrene.

In 2013, due to a severe cough caused by a lung infection, accompanied by chest and back pain, my lung was atomized with a solution of tea and sodium chloride. The solution was inhaled through the mouth and exhaled through the nose, after five minutes, the coughing stopped, and a large amount of phlegm was expelled after ten minutes. Thirty minutes later, the chest and back pain subsided. This discovery sparked my interest as it represents a new method for lung cleaning.

From 2013 to 2016, experiments were conducted with nearly a hundred different plants and sodium chloride, including Banlangen, Huangqi, Lianqiao, Mulberry Leaf, Ginger, Goji Berry, Chrysanthemum, Sicklepod Seed, and Poria. The experiments involved atomizing with solution including the plants and sodium chloride into the lungs and mouth. However, the results were unsatisfactory, and experiments had inflammatory reactions, such as atomizing the lung with solution including Banlangen for thirty minutes, no mucus was expelled but neck pain was caused. Then, the lung was atomized with a solution of tea and sodium chloride, mucus was expelled. Through the atomization, the neck pain was eliminated. At the same time, the formulation solution was dropped into the nasal cavity and a linear conduction of electric current from the nasal cavity to the trigeminal nerve was found. Using the formulation solution in the oral cavity, there was numbness in the mouth, and a ‘current’ was felt in the neck area and inflamed areas of the teeth. The wounds caused by scratches, mosquito bites, and burns were soaked in the formulation solution. A spreading ‘electric current’ stimulation was felt in the wounds, which was disappeared after one minute. The pain, itching, and swelling be disappeared after ten minutes.

In 2016, the laws governing the experiments of the previous three years were summarized. The formulation solution was absorbed by the nervous system and the conduction of ‘electric current’ was felt. The felt current is stronger in severe inflammation, while it is weaker in mild inflammation. Mucus was expelled at a fixed position, with more mucus expelled in severe inflammation and less in mild inflammation. As the mucus is expelled, the pain is alleviated.

From 2016 to 2021, considering the difficulty of conducting animal experiments due to the inability to determine the location of pain in animals, the inability to observe mucous secretion in the animal's digestive tract, and the inability to observe the fixed path of the animal, experiments had to be conducted on researchers' own bodies. Particularly in experiments involving the eyes, brain, spinal cord, and heart, only when researchers personally participate can original test data be obtained and drug resistance and safety truly verified.

The researchers have conducted tens of thousands of experiments on themselves, which included the immersion experiments carried out using solutions on the corresponding parts of the eyes, nose, mouth, nipples, genitals, rectum and skin, microinjections of the solution into the scalp, injections of the solution into the skin throughout the body, and injections of the solution into the lump in the neck. In recent years, twenty or thirty relatives and friends have participated in the test, and the characteristics and effects of the tests are very close.

The embodiments of the present application provide a technical solution to solve the above technical problems with the following general idea:

As mentioned above, there is an urgent need to develop effective preventive and therapeutic measures for aging. The core of this application is to find a method of using the nervous system for drug delivery, while inhibiting inflammation and tumor growth through nervous system drug delivery, achieving regeneration and repair of damaged areas. The absorbed substances are converted into neurotransmitters by the nervous system rapidly. The pain signals can be easily blocked by electrical signals involving saturated sodium ions. These electrical signals are transmitted to the immune system, where, in synergy with the nervous system, the inflammatory substances and mucus are rapid eliminated. Therefore, by implementing drug delivery through the nervous system and simultaneously eliminating inflammatory mucus, the efficiency of disease suppression is significantly enhanced, along with an improved efficiency in pain inhibition.

Based on the spiral curve changes in sodium levels, it is speculated that the range of 440 mmol/l to 451 mmol/l is an important region for regeneration. Beyond this 440-451 range, regeneration may be triggered, eliciting almost simultaneous responses from the nervous system and the immune system.

According to a study by researchers at the Vanston Medical Research Institute in the United States, it was found that there is complex communication between two intricate biological systems: the nervous system and the immune system. The immune system response is controlled by neurons responsible for transmitting pain signals, with approximately 20 neurons innervating a lymph node. Researchers conducted human experiments by increasing the frequency and amount of the formula solution to elevate the level of electrical signals, in order to determine a fixed pathway for nervous system drug delivery. The timing of nervous system drug delivery and the location of mucus secretion were observed.

Based on implementation examples and experimental data, a detailed explanation of the inhibitory effects of the formula on diseases is provided below.

### The First Embodiment

This embodiment uses the solution prepared from Tieguanyin tea and sodium chloride to conduct experiments on the inhibitory effects of inflammation, including the following specific steps:

Step 1, Preparation of the formulation solution: in a 316 stainless steel equipment, boil 2 kg of water, then add 400 g of Tieguanyin tea into the boiling water, continue to boil for five minutes, and then steep for twenty minutes (the measured temperature of the tea at this point is 90 degrees Celsius); at this point, the concentration of the tea in the water is greater than 40 mg/ml (typically as the first brew of tea). In a glass or ceramic container, add 600 g of sodium chloride, pour in the first brew of tea for mixing, and let it cool to room temperature for use.

### Step 2, Human Experiment:

### Oral Experiment

Fixed Pathway: Oral cavity, tongue to dental nerve to neck to stomach;

Characteristics: the mucus is expelled and the mouth is numb; 10-20 milliliters of solution are placed in the mouth, held for 2-5 minutes, and then spit out, with mucus expelled in the throat; oral numbness is also present. At the beginning of use, the electric signal can be felt reaching from the oral cavity to the dental nerve to the neck and then to the stomach. If the inflammation is strong, the feeling will be strong; if the inflammation is weak, the feeling will be weak or there will be no feeling at all. When used 1-2 times a day for more than two weeks, loose teeth gradually become tight, missing enamel is regenerated and repaired, and gum muscles are increased and thickened.

Oral Experiment: Within four hours, more than ten consecutive oral experiments were conducted, and it was observed that the oral mucosa appeared white with increased mucosal folds. Initially, it may give the sensation that the oral mucosa is about to peel off, but after 10 hours, the folds reduced and 70% of the mucosa regained its pink color. After 24 hours, the mucosal folds completely disappeared, and 100% of the mucosa returned to normal. It is safe to use the formulation solution on the mucosa.

Case: a relative of the researcher was admitted to the emergency room for respiratory failure and required intubation for assisted breathing. After four days of intubation, the patient developed foul breath, multiple ulcerations on the tongue and oral mucosa, and significant ulceration around the mouth. Use 10 milliliters of formulation solution and hold it in the patient's mouth for five minutes before suctioning it out. Then, clean the mouth with 10 milliliters of water. Use the formulation solution four times a day for 24 hours by soaking the mucosa in the patient's mouth and then suctioning it out. It was observed that the oral odor significantly decreased after use, and after 24 hours, scabs formed on the skin ulcers outside the mouth. Ten days after intubation, the scabs around the mouth fell off, and the oral mucosa was completely repaired to normal.

The patient's teeth were loosened, and the formulation solution was used to soak the oral cavity 1-2 times a day, with 10-20 milliliters of solution each time, staying for 5 minutes, and continuing for four weeks. It was observed that after two weeks, the gingival recession had alleviated, there was an increase in both the mass and thickness of the gingival muscles, the height and thickness of the gingival muscles had increased, enamel regeneration and repair had occurred, and the oral odor had become mild. Observations revealed that daily use of the formulation solution conformed to the regular spiral curve of sodium ions, leading to the repair and regeneration of teeth and gums.

### Eye-Drop Experiment:

Fixed Pathway: from the eyes to the occipital bone to the sacrum to the heart;

Characteristics: the mucus is expelled and the pain is suppressed; drops on the lower and upper eyelids are more efficient; eye pain, head pain and spinal pain are relieved; drainage of mucus from meningeal lymphatics is accelerated; some mucus is drained from the anterior and posterior pharyngeal wall position near the occipital sinus; more inflammation leads to more mucus, less inflammation leads to less mucus.

Observe 1-2 times daily; when administering eye drops, use your finger to pull down the lower eyelid and administer the drops for one to two minutes. Then, use your finger to pull up the upper eyelid and administer the drops for one to two minutes. After administering the drops, inflammatory substances from the eyes will be discharged. The participants in the experiment all experienced a sense of pain at the beginning, which gradually disappeared after one minute. After four weeks of eye drops, the heart would follow the enhanced beating with each drop, and the heart returned to a steady state at the end of the drops. The relief of pain in the eyes and brain is relatively quick after administering eye drops; eye drops lead to a rapid reduction in brain swelling, and have a noticeable effect in expelling inflammatory substances from the brain. After continuous administration of eye drops, there is an increase in the muscles around the eyes. The orbicularis oculi and temporal muscles show significant improvement, and wrinkles around the eyes decrease. After continuous administration of eye drops, there is a noticeable improvement in cardiac function. The heart rate is not significantly elevated during physical activity, and overall physical performance is enhanced.

The researchers observed that mucus was discharged near the posterior pharyngeal wall and occipital sinus after administering eye drops. After the mucus was expelled, brain inflammation and pain were immediately relieved or disappeared.

In the eye drop experiment, observe the lower eyelid and upper eyelid positions and administer approximately 0.02 milliliters of the formulation solution each time. The researchers administered eye drops every half hour, with over 20 eye drop administrations within 24 hours. The cumulative effect of multiple eye drop administrations resulted in an enhancement of electrical signals, and participants clearly felt the instantaneous transmission of electrical signals through a point-to-point jumping method from the eyes to the occipital bone, from the occipital bone to the sacrum, and finally to the heart. It only takes about 0.6 seconds for the electrical signals to travel from the eyes to the heart using this point-to-point jumping method, which can continue for more than 24 hours.

After the researchers administering eye drops more than 20 times a day for consecutive days and continuously for seven days, participants could feel the continuous point-to-point jumping method of transmission from the eyes to the occipital bone, from the occipital bone to the sacrum, and from the sacrum to the heart when their eyes were closed. The continuous strong signals had a significant impact on sleep, gradually leading to fatigue, weakness, and muscle soreness, but without other discomforts. Recovery could occur quickly after stopping the eye drops for three to four days. It is speculated that the concentration of sodium ions absorbed by the nervous system increases with the eye drops. Meanwhile, the rate at which sodium ions are metabolized is relatively slower than the absorption rate, leading to an excessive accumulation of sodium ions in the nervous system. Normal levels can be restored after three to four days of stopping the eye drops.

### Case:

From 2008 to 2012, the researcher experienced headaches and underwent hospital examinations which indicated that the headaches were caused by abnormal brain blood vessels. They observed a progression from experiencing headaches once or twice a month to having them once or twice a week. Additionally, they noticed feeling lightheaded while walking. Subsequent hospital examinations revealed lacunar cerebral infarction with two cavities or holes in the left and right sides of the brain. After receiving intravenous injection at the hospital for 5 minutes, the headache worsened and the pounding pain in the brain intensified. The intravenous injection was discontinued, and the individual went home to rest and recover. Subsequently, he tried a fasting therapy by restricting diet, which provided some relief from the head pain, resulting in experiencing headaches once or twice a month. Then, in 2016, he started using a formulated mist for eye nebulization and eye drops.

Observations revealed that after daily continuous eye drops, the symptoms of headaches disappeared almost completely. There was no need to restrict the diet, and sleep patterns returned to normal. A new brain CT scan showed the disappearance of lacunar cerebral infarction in both the left and right sides of the brain, indicating the repair of the cavities or holes in the brain. After years of using eye drops, heart tests showed normal results. A noticeable change was the reduced intensity of heartbeats during intense activities like running. The heart function significantly improved, along with increased endurance. Observations revealed that daily use of the formulation solution resulted in the repair and regeneration of brain inflammation and damage, which complied with a regular sodium ion spiral curve.

The family member of the researcher was hospitalized in the emergency department due to subarachnoid hemorrhage that broke into the brain ventricle, resulting in brain swelling and respiratory failure. Observations of the patient's eyes revealed blurred boundaries between the iris and sclera, along with severe bruising and blisters on the eyes. The formulation solution was used for eye drops, with a dosage of 0.04 milliliters applied to the lower eyelid for approximately 2 minutes each time. Observations revealed that after 24 hours, a significant amount of inflammatory proteins appeared from both the upper and lower eyelids. Eye drops were administered approximately ten times a day, and it was observed that a substantial amount of inflammatory proteins were expelled from the eyes daily. After continuous eye drops for ten days, the sclera recovered, and the pupils and iris regained brightness. The patient's brain swelling was eliminated.

Multiple middle-aged and elderly individuals participated in the experiment, with daily continuous eye drops. Observations revealed that after the eye drops, there was an increase in the muscles around the eyes, including the orbicularis oculi and temporalis muscles, and a reduction in periorbital wrinkles. Additionally, it was observed that along with the eye drops, there was an improvement in heart function and exercise capacity.

A close relative had a degenerative eye disease, which included retinal detachment, vitreous opacity, and macular hole, resulting in severe distortion of vision that required surgical treatment. The patient voluntarily participated in the eye drop experiment and did not use any other medications during the process. Observations revealed that three months after using the eye drops, the distortion of vision disappeared, and the visual acuity returned to normal. One year later, eye examinations showed a clear vitreous body, a normal retina, and improvement in the macular hole. It was observed that daily use of the formulation solution led to a regular sodium ion spiral curve, resulting in the repair and regeneration of eye inflammation and eye damage.

### Nasal Experiment:

Fixed pathway: From the left and right sides of the nose to the trigeminal nerve to behind the ear; from the posterior nasal mucosa to the position of the frontal cerebral cortex.

Characteristics: Linear conduction, mucus secretion, pathway from the nasal side to the trigeminal nerve, repair of rhinitis and nasal damage; administer 1 milliliter of solution in multiple doses to the left and right nasal cavities, positioned 2 centimeters from each nostril; observations revealed that the electrical signal linearly conducted from the nasal side to the trigeminal nerve behind the ear; mucus secretion occurred within the nasal cavity, starting around 1 minute after administration, with effects including the inhibition of nasal inflammation, pain, and trigeminal neuralgia; it was observed that deep nasal administration up to ten centimeters reached the mucosal position, instantly conducting to the frontal cerebral cortex location, causing a gradual relief of numbness after about one minute, accompanied by a significant outflow of mucus from the nose.

### Case:

When drops to the nasal side firstly administered by the researcher, a spicy sensation at the nasal position was felt, with the “electric current” linearly and slowly moving towards the direction of the trigeminal nerve, leading to a significant outflow of nasal mucus; the “electric current” persisted for about 2 minutes. After 24 hours, a second nasal administration resulted in a continued slow movement of the “electric current” towards the trigeminal nerve, accompanied by a large outflow of nasal mucus, which disappeared after 2 minutes. On the third day of nasal administration, the speed of the “electric current” increased, reaching the trigeminal nerve and then rotating into the cochlea, leading to a reduction in nasal mucus. In the following days, the signal linearly conducted when administering drops to the nasal side but was no longer as intense, with a decrease in mucus production. After one month, there was almost no sensation upon nasal administration, and the amount of mucus was reduced. Observations revealed that daily use of the formulation solution resulted in the repair and regeneration of nasal inflammation and nasal damage which complied with a regular sodium ion spiral curve.

### Rectal Experiment

Fixed pathway: rectum to perianal nerves to sacral plexus to spinal column; rectum to large intestine to small intestine to stomach to neck.

Characteristics: mucus was discharged, waste gas emission, urination, and defecation functions were improved. Observations revealed that rectal administration of 10 milliliters of solution resulted in waiting for one minute, followed by continuous flatulence, strong intestinal twisting, and the initiation of defecation and urination; Each rectal administration of the formulation solution resulted in the discharge of mucus to lubricate the intestines, without causing anal fissures; Approximately 30 milliliters of foul-smelling transparent mucus would be discharged from the rectum after one to two months; Observations revealed that defecation became easier with the use of the formulation solution, with a distinct feeling of exertion at the sacrum position. Prior to using the formulation solution, the individual would exert force throughout the body during defecation, including the neck and scalp, but after using the formulation solution, only the lower back required exertion, with no force being applied to the head.

### Case:

The participants in the experiment had rectal polyps, hemorrhoids, and stomach problems. During the first rectal experiment, there was a longer flatulence time, and there was some pain when the colon twisted, with defecation and urination occurring after one minute, and the feeling of colon twisting ending after defecation. Observations revealed severe inflammation in the digestive tract, with the electrical signal linearly conducting from the rectum to the stomach and neck. After continuing to use the formulated solution for two to three days, there was no longer pain when the colon twisted, and flatulence, urination, and defecation were smooth without any sensation of an electrical signal transmitting upwards. After using the solution for three months, observations revealed that the hemorrhoids had shrunk, the rectal polyps had disappeared, and the perianal inflammation had subsided.

Many elderly people participated in the rectal experiment and used the formulation solution daily, resulting in a large amount of waste gas emission, smooth urination, and easy defecation. Observations revealed that using the formulated solution daily resulted in the regular spiral curve of sodium ions, allowing for the repair and regeneration of perianal inflammation and rectal damage.

### Lung Experiment

Fixed pathway: From the mouth and nose to the lungs, to the neck area.

Characteristics: large amount of mucus was discharged, pain in the chest back and neck was relieved. The atomization process lasted about 30 minutes using 10-20 ml of solution. The atomization process involves inhaling through the mouth and exhaling through the nose to allow the mist to reach deep into the lungs. Most people will start coughing and expectorating around 7 minutes of atomization, as the pulmonary sensory receptors (such as cough receptors) and the immune system work together to expel mucus, transporting it to the throat for active expectoration. After half an hour of atomization, approximately 40-60 milliliters of mucus can be expelled, and atomization can be continued multiple times a day to expel inflammatory exudate from the lung.

### Case:

The researcher infected with pneumonia experienced lower back pain, neck pain, and headaches. The headache was relieved after eye drops, but returned two hours later. Therefore, continuous lung atomization was performed to expel 60 milliliters of phlegm, while eye drops were used to eliminate the headache. After 24 hours, atomization was performed twice a day, leading to the relief and disappearance of headaches, neck pain, and lower back pain.

Patients with pulmonary nodules participated in the experiment, with a majority being elderly women. Observations revealed that most elderly individuals experienced neck pain and anterior chest and back pain. After continuous atomization to expel mucus from the lung daily, the pain disappeared. Some elderly individuals with asthma and pulmonary tuberculosis underwent long-term atomization, starting with daily sessions. After significant improvement, they reduced atomization to two or three times a week for a week, followed by one or two sessions per month.

### Skin Experiment:

Fixed pathway: From the epidermis to the subcutaneous nerve receptors.

Characteristics: the skin inflammation can be inhibited without scarring by topical application and soaking, while subcutaneous injection of micro-needles can inhibit pain. The defective area was repaired by regeneration. Observations had shown that in cases of burns, cuts, and scalds, a strong sensation is instantly produced when applying and soaking the wound site with the formulated solution. One minute later, the burning sensation gradually dissipates, with no pain after ten minutes. After 24 hours, there is no inflammation at the wound site. Subsequently, scab formation begins, and after the scab falls off, no scars are left behind.

Observations have revealed that when the solution was applied to facial skin and massaged with rotating fingers, the skin becomes warm and red. It has been observed that in areas with severe skin and muscle necrosis, soaked in the formulated solution for over an hour results in a significant amount of inflammatory substances detached. With continuous soaked for five to six hours, the wound does not hurt, and all infected inflammatory substances are shed, allowing for natural wound healing. Ten hours later, scab formation occurs at the wound site. Subsequently, applying the solution around the area daily for eight weeks will lead to regeneration and repair of the missing muscle and skin tissue.

### Case:

The researcher's calf was scratched by a tile, and disinfection was done using iodine and phenol. However, an infection occurred. After 4 weeks, the wound area expanded to approximately 4 square centimeters, and a pit formed at the site of muscle suppuration with a depth of 0.8 centimeters. The researcher poured the formulated solution into the pit and the skin was soaked continuously for over 6 hours, resulting in a significant shedding of inflammatory substances. After 24 hours, some inflammatory substances were still visible. The skin was continued to be soaked for another six hours, and the inflammatory substances was shed on their own. It was observed that 10 hours later, scab formation was occurred, and then the scab was elevated. At the edge of the pit, a transparent scab, four millimeters wide, was appeared. After 24 hours, a dark red liquid was appeared beneath the transparent scab. Subsequent drying elevated the scab, and the dark red liquid was continued to be appeared beneath the transparent scab after each drying. The scab was continued to be elevated and thickened. After 8 weeks, the scab was fell off, and the missing muscle and skin were repaired and regenerated. Observations revealed that daily use of the formulated solution followed a regular spiral curve of sodium ions, achieving repair and regeneration of skin inflammation and muscle damage.

The researcher had been bald on the top of the head for twelve years, always maintaining a flat hairstyle. Before injection, no pain was sensed when pricking the scalp. Micro-needle injections of the formulated solution was conducted, with each injection being 0.01 milliliters, at over twenty positions on the top of the head. The injections were administered once a week for four consecutive weeks. Observations revealed that there was a sensation of pain when pricking the scalp, and hair growth began on the top of the head. It was speculated that the baldness was caused by the disappearance of nerves near the scalp hair follicles. However, a significant number of nerve receptors surrounding the hair follicles on the scalp were repaired and regenerated by micro-needle injections, leading to natural hair regrowth. Observations also showed that daily use of the formulated solution followed a regular spiral curve of sodium ions, achieving repair and regeneration of damaged nerve receptors around the hair follicles.

A senior in the circle of friends and family suffered from lumbar disc herniation, experiencing severe pain and inability to walk, resulting in a weight loss from 100 pounds to 80 pounds. They received four micro-needle injections of 0.01 milliliters of the formulated solution at the painful site, and it was observed that the pain immediately eased, allowing the individual to get out of bed and use the restroom. The senior used the formulated solution to clear the intestines daily and received injections of the solution every two days. After one month, the senior could walk freely, and the weight returned to 100 pounds. One year later, a hospital examination revealed the disappearance of the lumbar disc herniation. The senior now maintains daily intestinal clearance and receives micro-needle injections once a week. Observations also showed that daily use of the formulated solution followed a regular spiral curve of sodium ions. Through skin needle injections, partial repair and regeneration of lumbar inflammation and lumbar spine damage were achieved.

### Nipple Experiment

Fixed pathway: Nipple to thoracic dorsal nerve to cervical plexus to brachial nerve to radial and ulnar nerves to hand; nipple to cervical plexus to facial nerve.

Characteristics: inflammatory fluid is discharged, and joint flexibility is restored.

Each time, 20 milliliters of the solution is placed in a glass container and placed over the nipple, the nipple is soaked for about 20 minutes. Observations revealed that at the beginning of the soaking process, there were slight nerve tremors in the chest and neck. However, there was no sensation of pain in the nipple.

Observations revealed that soaking the nipple led to the restoration of flexibility in the neck area, chest and back scapula, with gradual elimination of arm pain, shoulder pain, and chronic inflammation. There was an increase in muscle mass around the face and ears, a gradual reduction of wrinkles behind the ears, and an increase in masseter muscle. Additionally, there was an increase in neck muscle mass and a reduction in wrinkles.

Observations revealed that nipples of women were soaked twice a day, leading to the discharge of breast fluid and gradual elimination of breast inflammation. The breast lumps in women began to be softened and broken down. After using the solution, some women's breasts became plumper, and the size of their bra cups increased.

### Case:

The nipples of the researcher were soaked, resulting in that: the inflammation in the neck area's nervous system was disappeared; the flexibility in the neck region was improved; neck lumps were gradually disappeared; wrinkles around the ears were decreased gradually; muscles behind the ears and in the face were increased gradually; the neck muscle mass was increased and wrinkles were reduced.

One participant in the experiment had fluid accumulation in one side of the breast and 7 breast lumps. The nipple was soaked twice a day with a 20-milliliter solution for twenty minutes each time. Observations after four weeks showed that the fluid was disappeared, the breast lumps became softer and smaller, and the arm pain was gradually decreased. It was observed that daily use of the formulation solution conformed to the regular spiral curve of sodium ions. Through soaking the nipple, partial repair and regeneration of breast inflammation and mammary gland damage were achieved.

### External Genitalia Experiment:

Fixed pathway: From the external genitalia to the sacral plexus, then transmitted through several paths;

from the sacral plexus to the lumbar plexus to the occipital bone to the eyes; from the sacral plexus to the sciatic nerve to the tibial nerve to the plantar nerve; from the sacral plexus to the superior and inferior gluteal nerves; from the sacral nerve to the pudendal nerve, dorsal nerve of the penis, perineal nerve, etc.; from the external genitalia to the sacral plexus, to the lumbar plexus, to the bladder, to the kidneys.

Characteristics: the motor function and the sexual function were enhanced, and the neural inflammation was eliminated.

Males can soak their external genitalia in a 20-milliliter solution in a glass container, while females can wrap their buttocks in transparent plastic wrap and sit on a chair. It was observed that soaking the external genitalia for 20 minutes each day with 20 milliliters of solution resulted in mild stimulation of the external genitalia mucosa and urethral mucosa, with an overall good sensation. The signal is transmitted linearly from the external genitalia to the sacral plexus to the lumbar plexus, with a signal strength stronger than that of eye drops, around 0.6 seconds each time.

Observations revealed that continuous soaking of the external genitalia and application of the skin around the external genitalia resulted in gradual strengthening of the muscles in the lower limbs, buttocks, and legs. This led to an improvement in walking and motor abilities, as well as an enhancement in sexual function. In males, hardness and endurance gradually improved, while in females, sensitivity of the sexual organs was restored.

The improvement in neural inflammation is very noticeable, as is the improvement in pain relief, such as sciatic nerve pain, lumbar disc pain, and testicular pain, which are reduced or eliminated. For patients with herpes simplex virus, the effect of virus clearance is significant.

### Case:

The external genitalia of the researcher was soaked twice a day, and after soaked, electrical signals were linearly transmitted from the sacral plexus to the lumbar plexus, then to the occipital bone and eyes. At the same time, there was a noticeable improvement in leg function, with increased walking speed. Sexual function also was improved significantly, with enhanced hardness and endurance.

After the external genitalia of an experimenter was soaked, a pain sensation from the occipital bone to the eyes was experienced as the electrical signal was traveled from the sacral plexus to the lumbar plexus and then to the occipital bone and eyes. The pain sensation was lasted for ten minutes. This situation indicates the discovery and repair of old damage in the nervous system through regeneration processes. After three days of being soaked, there was no longer any pain sensation in the eyes. Similar cases have been observed in the nasal area.

A herpes simplex virus patient who experienced outbreaks every month soaked their genitalia and did not have any herpes outbreaks thereafter. It was speculated that soaking the external genitalia may help in clearing the HIV virus and the herpes simplex virus. Observations had shown that daily use of the formulated solution, following the sodium ion spiral curve pattern, can partially repair and regenerate nervous system inflammation and damage through soaking the external genitalia.

Observations had revealed that soaking the external genitalia had a beneficial repairing effect on chronic prostatitis, cystitis, and kidney inflammation in elderly individuals.

### Safety, Drug Resistance, and Side Effects:

The researcher conducted experiments from 2013 to 2020, spanning a period of 7 years, without inviting family and friends to participate in order to ensure safety. Many people are familiar with the renowned traditional Chinese medicine practitioner Ni Haixia, who personally experimented with medicines and continuously explored new methods. He used salt and Aconitum plants to brush his teeth every day, causing numbness in the mouth and strengthening his teeth. However, after several years, Ni Haixia became increasingly emaciated and eventually died of organ failure. I personally speculate that the reason for this is: long-term use of sodium chloride and Aconitum components can have harmful effects on the human body's neurotransmitters, leading to abnormalities in electrical signals over time.

Let's look at another case:

Formulation solution was used to drop into the eyes of turtles, hamsters, zebra finches, and frogs. Eye dropping experiments are the fastest method of verification, with ten drops per day and 1 milliliter of solution used each time, dropped onto the animal's eye area, where it remains on the animal's eye. Observations had shown that after eye drops, dozens of frogs became limp and weak, losing their ability to feed and secrete mucus. Their skin wrinkled all over, and they eventually died a few days later. It was observed that after eye drops, hamsters experienced erratic heartbeats leading to sudden death. Turtles showed muscle atrophy around the eyes after continuous eye drops for a week, while zebra finches had accelerated heart rates after eye drops and died suddenly a few days later. It is speculated that the formula used in this application is only suitable for humans, as it is similar to human neurotransmitters. However, it is not suitable for frogs, turtles, hamsters, and zebra finches.

Using Longan leaves and sodium chloride for eye drops on animals, it was observed that the eye muscles of turtles increased in size and height, and their reproductive organs in the tail were soaked, resulting in the turtle being agile and in good condition. However, after eye drops, frogs became limp and weak, and eventually died. Hamsters experienced erratic heartbeats leading to death, while zebra finches died after eye drops.

From this experiment, it is speculated that the neurotransmitters of turtles are more compatible with solution including Longan leaves and sodium chloride, leading to the increase in eye muscle mass in turtles. It is conjectured that solution including Longan leaves and sodium chloride is only suitable for turtles, as it is closer to the neurotransmitters of turtles. However, it is not suitable for frogs, hamsters, and zebra finches.

Through studying the eye-drop experiments and analyzing the different neurotransmitter components in various species, it is speculated that each animal species may correspond to a specific plant component in terms of neurotransmitters.

Therefore, after ten years of experiments, the safety of the formula has been confirmed, and the characteristics of the formula remain stable without any signs of drug resistance.

Since 2018, experimenter has been using the formula for daily eye drops, nasal drops, mouthwash, rectal irrigation, external genitalia and nipple application and skin application, with each application lasting approximately 1-4 minutes. Experimenter generally tests liver and kidney function once a year, without restricting their diets, and is allowed to consume high-purine late-night snacks, fried snacks, sugary drinks, alcohol, and starches. The experimenter's weight has increased from 80 kg to 95 kg, but the test values of the liver and kidney function have been remained normal. The albumin index was 45.5 g/L, the value of glutamyl transpeptidase was 40 IU/L, the bilirubin index was 9.7 umol/L; the value of microglobulin was 1.66 mg/L, the value of creatinine was 70 umol/L, and the value of serum urea was 3.68 mmol/L. After a long-term human experiment, it is believed that the safety and resistance risk of the formula is very low. Therefore, starting from 2020, friends and family begun to participate in the experiment, and each person's experimental results have been similar.

### Side Effects:

After injection, the injected area may appear red and take about two months to disappear. For example, in cases of lumbar disc herniation, a micro-needle injection of 0.1 ml at three or four points can immediately suppress pain, with relief lasting more than twenty-four hours. Injections are administered every other day, and the injection site does not swell or become inflamed but may scab and leave red marks. These red marks typically take about two months to disappear.

Observations have shown that upon waking up, there is sometimes a sensation of electric currents in the toes and fingertips, originating from the sacrum, accompanied by slight body tremors for about a minute. This condition is considered a normal phenomenon and does not qualify as a side effect.

### Summary of the Experiment:

The human body absorbs the formula in a fixed pattern, administering it along a fixed pathway of the nervous system. Inflammatory substances are immediately metabolized and excreted in the form of mucus secretion. The entire process is precise and controllable and does not affect normal sleep and diet. The formula has a rapid pain-relieving effect and can significantly alleviate the suffering of severe patients. It is non-invasive, comfortable on initial administration, and has good patient compliance. It also reduces adverse reactions and prevents the stimulation burden on the gastrointestinal tract and liver. The formula has good resistance to drug tolerance in the human body.

Route of administration: Mainly through active absorption by the nervous system;

Administration time: Once in the morning and once in the evening, applied for 2-10 minutes to a fixed position in the mucosal area;

Single dose: Around 0.1-0.3 ml for the eyes; 0.1-0.7 ml for the nasal side; 20-30 ml for the oral cavity; 10 ml for the rectum; 5 ml for the lungs, etc.; 10-20 ml for the nipple; around 20 ml for the external genitalia; no specific dosage limit for skin application.

In the specific application, a formula for inhibiting aging and promoting regeneration and repair is provided. Wherein, the formula comprises tea and sodium chloride; the tea consists of any combination of one or more types of tea, such as wulong tea, green tea, white tea, yellow tea, dark tea, and black tea, etc.; amino acids and other small molecules in tea ingredients, in terms of types and proportions, meet the needs of various neurotransmitters within the human nervous system; types and proportions of small molecules, such as amino acids, in tea ingredients are in accordance with needs of various neurotransmitters within human nervous system; one of the formulas comprises Tieguanyin tea and sodium chloride; a solution of the formula is prepared as follows: dissolved ingredients of Tieguanyin tea in water until ingredients of the tea reaching or close to the highest dissolution concentration, adding sodium chloride to bring the solution to a state in which sodium ions and chloride ions are saturated (sodium chloride in the solution reaches its maximum solubility).

Tea is classified into various types, and one of the classifications is based on the degree of fermentation. There are approximately six major categories of tea based on this classification: the degree of fermentation of Oolong tea is 15%-30%, the degree of fermentation of green tea is 0%, the degree of fermentation of black tea is 100%, the degree of fermentation of white tea is 5%-10%, the degree of fermentation of yellow tea is 10%-20%, and the degree of fermentation of red tea is 70%-90%. The components of tea are quite similar, especially in the case of more than 20 types of amino acids which are quite similar. In experiments, teas with longer fermentation time showed poorer effects, while Oolong tea, green tea, etc., exhibited better and more stable effects. Over many years of testing, Tieguanyin Oolong tea has exhibited high stability. The therapeutic effect of tea lies in the fact that there are nearly 500 different types of components in tea, including over 20 types of amino acids with a stable proportional and concentration relationship. This relationship is similar to the composition and concentration of neurotransmitters in the human body. Therefore, after active absorption by the human nervous system, tea can produce stable proportional and concentration relationships of neurotransmitters.

There are over 500 components in the tea, including vitamins (C, B1, B2, B3, B5, B6, B11, B12, P1, H, A, D, E, K, etc.), amino acids (theanine, asparagine, serine, glutamic acid, glycine, tryptophan, valine, histidine, arginine, threonine, alanine, proline, cysteine, tyrosine, aspartic acid, lysine, isoleucine, leucine, phenylalanine, etc.), alkaloids (caffeine, theophylline, theobromine, etc.), tea polyphenols (catechins, flavonoids, anthocyanins, phenolic acids, etc.), inorganic substances (potassium, phosphorus, calcium, magnesium, iron, sulfur, aluminum, fluoride, etc.), organic acids, lipids (phospholipids, glycerides, sulfur lipids, glycolipids), pigments (chlorophyll, anthocyanin, carotene, xanthophyll, etc.), aromatic substances, enzymes, and a high-concentration solution of sodium chloride, known as a formulated solution in experiments. One of the examples of formula solutions is that in our experiment, we aim to make the concentration of Tieguanyin tea components as close to saturation concentration as possible, with a concentration above 40-70 mg/ml. The concentration of sodium chloride solution is approximately 26.47%, which is the saturation concentration. At this point, the solubility is about 36.

In the specific application, a formula for inhibiting aging and promoting regeneration and repair is provided, the formula is applied to fixed positions of organs such as the eyes, nose, mouth, nipples, genitals, rectum, skin, lungs, etc., along fixed paths of the human nervous system for non-invasive drug administration, in order to reach the nervous system throughout the body and achieve long-distance drug delivery to the human nervous system; methods of drug administration also include microneedle administration and atomization administration; the fixed paths for non-invasive drug administration include: the drug administrated to the eyes is delivered to the heart through the occipital bone and the sacrum; the drug administrated to the nose is delivered to the ear through the trigeminal nerve; the drug administrated by nasal and oral atomization is delivered to the lungs and cervical nerves; the mouth and rectum are the two ends of the digestive system, the drug administrated through the mouth is delivered to the dental nerves, the neck and the stomach; the drug administrated through the rectal administration is delivered to the stomach and the neck along the digestive system; the drug administrated through the rectal administration is delivered to the to the sacral plexus and the nerves surrounding the anus; the drug administrated through the external genitalia is delivered to the sacral plexus, lumbar plexus, sciatic nerve, plantar nerves, superior gluteal nerve, inferior gluteal nerve, sexual nerves, urinary bladder, kidneys, and so on; the drug administrated through the nipple is delivered to the thoracic dorsal nerve, brachial plexus, cervical nerves, ulnar nerve, hand, facial nerve; the drug administrated to the skin is delivered to the neuroreceptors under the skin.

On the neuronal cell membrane, there are some special proteins such as sodium pumps, sodium ion channels, calcium ion channels, potassium ion channels, acetylcholine receptors, glutamate receptors, etc., forming a unique electrical signaling system. The neuronal cell membrane is a lipid bilayer, with vesicles enclosing the components of the formulation solution, forming electrical signals at the synapse. During this process, due to the effect of saturated sodium ion concentration, the efficiency of the nervous system's electrical signaling system is greatly enhanced, and the neurotransmitters transmitted are stable. Therefore, it can maintain the continuous stability of the body's electrical signaling level.

In specific examples, non-invasive administration via the eyes, nose, mouth, nipples, genitals, and rectum were analyzed, as well as microneedle injection and nebulized pulmonary administration, achieving systemic administration to the nervous system. Compared with existing oral and injectable drugs, this method has better drug efficiency, lower dosage, lower inflammation response, and fewer side effects.

In the specific application, a formula for inhibiting aging and promoting regeneration and repair is provided according to the formula of claim 1, wherein, a solution in which sodium chloride reaches a saturated concentration and a concentration of tea is close to saturation can be prepared according to the formula; a formula solution with high-concentration can be safely applied within sodium ion channels of human nervous system, while safely increasing concentration of sodium ions inside and outside neuronal cell membrane; concentrations of sodium ions, chloride ions, and small molecules such as amino acids inside and outside the neuronal cell membrane are simultaneously elevated by the formula, which is crucial for regeneration of a body; the formula solution is actively absorbed by human nervous system and is converted into multiple types of neurotransmitters; by a stronger electrical signal level, inflammation, cancer, bacterial and viral infections can be effectively inhibited, and transmission of inflammatory pain signals can be blocked; the concentration of sodium ions outside the neuronal cell membrane in the human body is approximately 150 mmol/L, and concentration of sodium ions outside the neuronal cell membrane of regenerative animals such as octopuses and squids is around 440 mmol/L; it is speculated that if the concentration of sodium ions outside the neuronal cell membrane in the human body exceeds 440 mmol/L, a regeneration process may be triggered; sodium ion channels are most abundant in the nervous system, and safe regulation of sodium ion channels is crucial for functions of the nervous system; a concentration of Tieguanyin and a concentration of sodium chloride in the formula solution have reached their saturation point, resulting in a sodium ion concentration of 4524 mmol/L; a concentration of sodium ions in the malignant tumor cell environment is 451 mmol/L, and a concentration of sodium ions in the inflammatory cell environment ranges from 200 to 300 mmol/L; 4524>451>440>300>200>150; the formula solution with a sodium ion concentration of 4524 mmol/L which can be safely and continuously used to human tissue and organs can be used to inhibit malignant tumors a sodium ion concentration in which is 451 mmol/L and pains caused by malignant tumors, and to suppress inflammation and edema a sodium ion concentration in which is 200-300 mmol/L and pains caused by the inflammation; in the specific implementation, the concentration of sodium ions outside the neuronal cell membrane in the human body can be increased from 150 to nearly 4524 mmol/L and then decreased back to 150 mmol/L; the solution is regularly and cyclically used every day to establish a repair and regeneration pattern that complies with the sodium ion spiral curve; the concentration of sodium ions in the solution starts from 150, reaches 4524, decreases to 150, and then reaches 4524 again, repeating in a regular cyclic pattern; when the concentration of sodium ions outside the neuronal cell membrane in the human body reaches or exceeds 440 mmol/L which is the concentration of sodium ions in the octopus cells, regenerative functions of cells is triggered; a cumulative regenerative frequency is maintained in a regular pattern every day, achieving an effect close to the regenerative capacity of an octopus.

According to the literature, multiple studies have confirmed that changes in cell membrane potential are related to the activity of cell mitosis. An increase in intracellular cation concentration indicates a higher likelihood of cell malignancy. It has been found that the percentage of the concentration of Na+ shows a gradually increasing trend in normal tissues, benign lesions or transitional tumors, and malignant tumor cells, with values of 0.05%, 0.05%˜0.07%, and 0.15%˜0.2%, respectively. The concentration of sodium ions in tumors and surrounding tissues is 50%˜60% higher than that in normal tissues. In cancer cells, on the one hand, there is a higher concentration of sodium ions, and on the other hand, large amounts of various amino acids are absorbed. According to J.ameron's measurements, the concentration of sodium ions in normal extracellular fluid is 138˜145 millimoles per kilogram (mM), while in the extracellular fluid of malignant tumor cells, the concentration of sodium ions is 451 mM.

According to existing medical literature, cancer is considered an immune system disease. However, in recent years, many scientists have found evidence through research that the nervous system and the immune system have a special relationship. It is possible that the lymphatic system is controlled by the sensory nerves, with approximately one lymph node controlled by twenty neurons. Following this thought, as long as the formulation solution is absorbed by the human nervous system, the immune system can be simultaneously regulated. Therefore, in human experiments, many cases have been observed where fixed characteristics of mucus discharge occur. For example, After soaking the nipples with the formula solution, inflammatory fluid is discharged; after using the formula solution to clear the rectum, mucus is discharged; after using the formula solution in the oral cavity, mucus is discharged in the throat area; after using the formula solution for eye drops, inflammatory proteins are discharged from the eyes, mucus is discharged outward from the meningeal lymphatic ducts; mucus is discharged after using the formula solution for nasal drops; after soaking the genitals with the formula solution, inflammatory mucus is similarly discharged from the lymphatic system. In specific operations, the formulation solution can be administered to any cancer pain site in the nervous system through methods such as micro-needle injection, nebulization, and non-invasive drug delivery, thereby inhibiting pain, suppressing cancer cell growth, and reducing inflammation. The cases describe the regeneration and repair process of some degenerative brain diseases, eye diseases, and spinal diseases. Following the sodium ion spiral curve, using the formula 1-2 times a day can accumulate stable regeneration signals, potentially regenerating and repairing degenerative diseases of the brain, eyes, and spine. The regenerative ability of the formula mimics the sodium ion concentration of octopuses, with the difference being that octopuses maintain the concentration of sodium ions at 440 mmol/l, while the concentration of sodium ions of the formula exceeds 440 mmol/l once a day.

Through research, the formula can repair nerve axons, enhance myelin sheath wrapping capacity, strengthen neuronal signal transmission, repair and regenerate blood vessels, muscles, skin, etc. It also reduces inflammatory scars and avoids scars forming physical barriers to regeneration and repair.

In the specific application, the present application provides a formula for inhibiting aging and promoting regeneration and repair, which is according to claim 1. Wherein, the drug is administered noninvasively along a fixed pathway in the nervous system; accumulating number of times of the regeneration in a regular manner to suppress aging; the usage time and dosage are as follows: 1-2 times per day; for the eyes, the usage time is approximately one minute each time, and the dosage is 0.1-1 milliliters each time; for the nose, the usage time is approximately two minutes each time, and the dosage is 0.1-1 milliliters each time; for the mouth, the usage time is about 2 minutes each time, and the dosage is 5-10 milliliters each time; for the lung atomization, the usage time is about 30 minutes each time, and the dosage is 5-20 milliliters each time; for the reproductive organs, the usage time is about 10-20 minutes each time, and the dosage is 5-10 milliliters each time; for the nipples, the usage time is about 10 minutes each time, and the dosage is 10 milliliters each time; for the rectum, the usage time is about one minute each time, and the dosage is 5-10 milliliters each time; for the skin, the usage time is about 5-10 minutes each time, and the dosage is 60 milliliters each time; other methods, such as injection, involve minimal injection volumes of approximately 0.1 milliliters each time; for diseases which are more serious, the time for the non-invasive drug administration can be extended to several hours per day, with the nervous system taking in the drug actively and accelerating the inhibition of inflammation.

In the specific application, a formula for inhibiting aging and promoting regeneration and repair is provided, and the formula is synthesized through physical methods.

Through extensive experimentation, it has been confirmed that drugs synthesized through physical methods exhibit the most stable and long-lasting drug delivery effect. The purpose of synthesizing drugs using physical methods is to preserve the integrity of the components. Tea contains over 500 components, which, when combined with saturated sodium chloride solution, are actively absorbed by the human nervous system and transformed into neurotransmitters with a rich variety of types and stable proportion of ingredients. The study of human neurotransmitters is highly complex and detailed. According to the biologist Yan Ning, there are approximately 200 different types of human neurotransmitters, while some scientists believe there are around 1000 types. The experiments combining tea components and sodium chloride to mimic human neurotransmitters are still ongoing. Through extensive foundational experiments, we believe that the combination of tea components and sodium chloride, corresponding to human neurotransmitters, is safe. This is because tea contains a wide variety of amino acids, vitamins, fatty acids, and rich inorganic compounds, all of which are essential for neurotransmitters. Therefore, it is speculated that about three-quarters of the components in tea, around 400 types, participate in the active absorption process of the nervous system and are transformed into over 400 types of neurotransmitters. However, this is only a speculation based on foundational research, and further extensive work and time are required for refinement.

Various purification experiments were conducted, such as using tea polyphenols as a substitute for tea ingredients, but the results were unsuccessful. For example, in the atomization extraction method, the liquid was collected after atomization and it was observed that the liquid turned from golden yellow before atomization to a light yellow color approaching transparency after atomization. Furthermore, when conducting eye-drop tests, the effectiveness was significantly better using the solution before atomization compared to the solution after atomization, indicating that some components were reduced after atomization, resulting in a decrease in the efficiency of the nervous system. Therefore, maintaining a high concentration of the formula solution is beneficial for maintaining a better drug delivery effect.

The half-life of the amino acids in tea components is short, and the stability can be prolonged by the saturated concentration of sodium chloride. According to current experimental observations, the newly prepared formulation solution has an efficacy of 100%. After one week, the efficacy decreases to 80%, and after two weeks, it drops to 60%. After three weeks, the efficacy decreases to around 40%, and it remains at 40% after four weeks. After five weeks, the efficacy drops to 30%, and after eight weeks, it remains at 30%. Here, the efficacy refers to the effect of controlling mucous secretion in human body experiments.

In the specific application, this application provides a formula for inhibiting aging and promoting regeneration and repair, used in the preparation, prevention, and/or treatment of the inflammation of the nervous system, the inflammation of the immune system and the degenerative disorder of the nervous system.

In specific applications, this application provides an application of the formula for inhibiting aging and promoting regeneration and repair. The inflammation of the nervous system, the inflammation of the immune system and the degenerative disorder of the nervous system include: pain from cancers of any type and other inflammatory pains; cancers and inflammations associated with central nervous system and immune system; degenerative disorders of the central nervous system, such as Parkinson's disease, Alzheimer's disease, amyotrophic lateral sclerosis, depression, insomnia, impairments and declines of functions of the autonomic and peripheral nervous system, impairments and declines of eye function, retinal degeneration, impairments and declines of brain functions, impairments of memory functions, impairments and declines of functions of the spinal cord and spine, impairments and declines of the functions of internal organs such as the heart, lungs, kidneys, impairments and declines of muscle functions, impairments and declines of the function of teeth and gums, hair loss caused by scalp neuritis; impairments and declines of the intestinal motility and defecation function; impairments and declines of the urination function of the urinary system; impairments and declines of sexual function, impairments and declines of the function of the skin.

In the field of cancer treatment, existing techniques such as chemotherapy, radiation therapy, and immunotherapy have been utilized. However, the mortality rate among patients remains high, particularly with low 5-year survival rates for malignant tumors. Most medical researchers believe that cancer is a disease of the immune system, where the immune system fails to recognize and control the spread and migration of malignant tumors. Cancer has a significant impact on the quality of life of elderly individuals, making it difficult to maintain a satisfactory standard of living.

Therefore, effective methods and treatment measures to inhibit aging are urgently needed.

In this application, the approach of simultaneously increasing the concentration of sodium ions and various amino acids is taken to develop means to inhibit diseases, which can suppress the development of inflammation and tumors from the initial stage of inflammation to the malignant tumor stage.

Simultaneously increasing the concentration of sodium ions and various small molecules such as amino acids outside the neural cell membrane in the human body to achieve or exceed the concentration of sodium ions inside and outside the neural cell membrane of octopuses, can enhance the regenerative ability of humans. It is speculated that cancer cells, especially malignant tumors, simultaneously increase the concentration of sodium ions and compete for the absorption of various amino acids. Malignant tumors absorb more than ten times the amount of amino acids compared to normal cells, with the aim of controlling the human nervous system to achieve regeneration, spread, and migration of cancer cells. Therefore, immunotherapy alone cannot completely suppress the migration and development of cancer cells. The inhibition of cancer must be achieved through the combined action of the nervous and immune systems. For example, the percentage of concentration of sodium ions shows a gradually increasing gradient trend in normal tissues, benign lesions or transitional tumors, and malignant tumors, with values of 0.05%, 0.05%˜0.07%, and 0.15%˜0.2% respectively. The concentration of sodium ions in tumors and the surrounding tissues is 50%˜60% higher than that in normal tissues. The concentration of sodium ions in the extracellular fluid of malignant tumor cells is 451 mmol/l, while the concentration of sodium ions outside the neural cell membrane of regenerative animals like octopuses is 440 mmol/l; these two concentration of sodium ions are relatively close.

To inhibit cancer cells, it is possible to achieve this by injecting the formulation solution into the central region of the tumor or soaking the tumor or designated absorption sites in the neural system with the formulation solution. When the formulation solution enters the center of the tumor, it causes the concentration of sodium ions of extracellular tumor cells to reach 4524 mmol/l, thus disrupting the physiological environment of malignant tumors. At the same time, the formulation solution is actively absorbed by the neural system, interacts with the immune system, and can facilitate the painless and continuous discharge of mucus, eliminating the contaminated fluids from the tumor. Additionally, the electrical signal intensity is much stronger than that of the tumor, thereby inhibiting the migration and growth of malignant tumors.

Malignant tumors simultaneously elevate the concentrations of various amino acids and sodium ions, approaching the extracellular concentration of neural cell membranes in octopuses at 440 mmol/l, which bears a significant resemblance to the findings analyzed by researchers on regeneration. It is speculated that the mechanism of malignant tumors also involves controlling the human nervous system and subsequently regulating the immune system. The sodium ion concentration in the extracellular fluid of malignant tumor cells is 451 mmol/l which is very close to 440 mmol/l that is the extracellular concentration of sodium ions in octopus neural cell membranes. Malignant tumors can be seen as “another form of life”. The mechanism of cancer cells in malignant tumors involves increasing levels of both sodium ions and amino acids. The extracellular fluid of malignant tumor cells at 451 mmol/l is much higher than the extracellular concentration of sodium ions in human neural cell membranes at 150 mmol/l, enabling easy control of the human nervous system, facilitating the ability to spread and metastasize rapidly throughout the body.

On the contrary, when the formulation solution is absorbed by the human nervous system, the concentration of sodium ions outside human neural cell membranes reaches 4524 mmol/l. In theory, the concentration of sodium ions inside human neural cell membranes would instantly reach 452.4 mmol/l, while the extracellular fluid concentration of cancer cells is 451 mmol/l. At this point, cancer cells are completely unable to control the human nervous system. Because long-term and extensive human experiments have confirmed that the formula is almost harmless to human healthy tissues, humans can utilize the formulation solution to soak specific areas such as the eyes, nipples, and genitals to suppress pain, inhibit cancer, and accelerate the discharge of mucus. Additionally, the use of injection methods can disrupt the cancer cells' living environment from within the tumor. As a result, the human nervous system can control cancer cells in a reverse manner, which aligns with the principle of natural selection in biological systems.

Cancer, primarily targeting mucous tumors, solid tumors, and malignant tumors can be suppressed by the formulation solution, such as brain cancer, lung cancer, liver cancer, kidney cancer, prostate cancer, thyroid cancer, pancreatic cancer, colon cancer, cervical cancer, skin cancer, central nervous system-related cancers, melanoma, lymphoma, oral cancer, etc.

Delivering drugs through the nervous system has many advantages. The inflammation of the nervous system can be suppressed by through the non-invasive administration, injection, and nebulization of the formulation solution. It has been found that a lymph node is controlled by twenty neurons in current technological discoveries. The nervous system is closely related to the immune system. The inflammation of the nervous system and the inflammation of the immune system can be suppressed by formula, while the discharge of the mucus and pain suppression are also promoted. Additionally, in experiments on multiple tissue organs, muscle growth has been achieved. For example, there is muscle growth around the eyes, gum muscles. Soaking of external genitalia enhances the sacral plexus nerve signal levels, gradually leading to growth of buttock and thigh muscles. Sexual organ function has been restored, and eye function damage has been repaired. After a decade of experiments, the safety of the formulation has been progressively confirmed.

In the specific application, a formula for inhibiting aging and promoting regeneration and repair is provided. A drug for prevention and/or treatment of the inflammation of the nervous system, the inflammation of the immune system and the degenerative disorder of the nervous system is provided, and active ingredients of the drug include the formula as described above; a dosage form of the drug is any one of a spray, a liquid preparation, an injectable preparation, a tablet, a capsule, a granule, a suspension mixture and a pill.

Analysis of the industrial practicality: the saturated concentration of sodium chloride is approximately 26.47%, and at this concentration, the solubility is around 36. The use of saturated concentration of sodium chloride provides stability assurance for industrial practicality. Although the half-life of the amino acids is short, they can be significantly prolonged through the saturated sodium chloride. This leads to a substantial extension of the efficacy and disease-inhibiting capabilities of small molecular components of amino acids. The clear composition of sodium chloride in tea ensures stable medicinal effects and meets the requirements for preparing medicines.

The formulated drug is made from natural products with very high stability, thus no preservatives or stabilizers are required. It is easy to be produced and can be stored for several months after production. However, researchers conducting replication experiments are advised to use formulation solution stored for approximately one week for optimal results in regenerative experiments.

Splitting, adding variations or substitutions to the formulation that can be readily thought of by any person skilled in the art within the technical filed of the application as demonstrated herein should be covered by the scope of protection of the application. Therefore, the scope of protection of the application should be based on the scope of protection of the claims.

1. A formula for inhibiting aging and promoting regeneration and repair, wherein, the formula comprises tea and sodium chloride; the tea consists of any combination of one or more types of tea, such as wulong tea, green tea, white tea, yellow tea, dark tea, and black tea, etc.; amino acids and other small molecules in tea ingredients, in terms of types and proportions, meet the needs of various neurotransmitters within the human nervous system; types and proportions of small molecules, such as amino acids, in tea ingredients are in accordance with needs of various neurotransmitters within human nervous system; one of the formulas comprises Tieguanyin tea and sodium chloride; a preparation method of a solution of the formula is as follows: dissolved ingredients of Tieguanyin tea in water until ingredients of the tea reaching or close to the highest dissolution concentration, adding sodium chloride to bring the solution to a state in which sodium ions and chloride ions are saturated (sodium chloride in the solution reaches its maximum solubility).

2. The formula according to claim 1, wherein, the formula is applied to fixed positions of organs such as the eyes, nose, mouth, nipples, genitals, rectum, skin, lungs, etc., along fixed paths of the human nervous system for non-invasive drug administration, in order to reach the nervous system throughout the body and achieve long-distance drug delivery to the human nervous system; methods of drug administration also include microneedle administration and atomization administration; the fixed paths for non-invasive drug administration include: the drug administrated to the eyes is delivered to the heart through the occipital bone and the sacrum; the drug administrated to the nose is delivered to the ear through the trigeminal nerve; the drug administrated by nasal and oral atomization is delivered to the lungs and cervical nerves; the mouth and rectum are the two ends of the digestive system, the drug administrated through the mouth is delivered to the dental nerves, the neck and the stomach; the drug administrated through the rectal administration is delivered to the stomach and the neck along the digestive system; the drug administrated through the rectal administration is delivered to the to the sacral plexus and the nerves surrounding the anus; the drug administrated through the external genitalia is delivered to the sacral plexus, lumbar plexus, sciatic nerve, plantar nerves, superior gluteal nerve, inferior gluteal nerve, sexual nerves, urinary bladder, kidneys, and so on; the drug administrated through the nipple is delivered to the thoracic dorsal nerve, brachial plexus, cervical nerves, ulnar nerve, hand, facial nerve; the drug administrated to the skin is delivered to the neuroreceptors under the skin.

3. The formula according to claim 1, wherein, a solution in which sodium chloride reaches a saturated concentration and a concentration of tea is close to saturation can be prepared according to the formula; a formula solution with high-concentration can be safely applied within sodium ion channels of human nervous system, while safely increasing concentration of sodium ions inside and outside neuronal cell membrane; concentrations of sodium ions, chloride ions, and small molecules such as amino acids inside and outside the neuronal cell membrane are simultaneously elevated by the formula, which is crucial for regeneration of a body; the formula solution is actively absorbed by human nervous system and is converted into multiple types of neurotransmitters; by a stronger electrical signal level, inflammation, cancer, bacterial and viral infections can be effectively inhibited, and transmission of inflammatory pain signals can be blocked; the concentration of sodium ions outside the neuronal cell membrane in the human body is approximately 150 mmol/L, and concentration of sodium ions outside the neuronal cell membrane of regenerative animals such as octopuses and squids is around 440 mmol/L; it is speculated that if the concentration of sodium ions outside the neuronal cell membrane in the human body exceeds 440 mmol/L, a regeneration process may be triggered; sodium ion channels are most abundant in the nervous system, and safe regulation of sodium ion channels is crucial for functions of the nervous system; a concentration of Tieguanyin and a concentration of sodium chloride in the formula solution have reached their saturation point, resulting in a sodium ion concentration of 4524 mmol/L; a concentration of sodium ions in the malignant tumor cell environment is 451 mmol/L, and a concentration of sodium ions in the inflammatory cell environment ranges from 200 to 300 mmol/L; 4524>451>440>300>200>150; the formula solution with a sodium ion concentration of 4524 mmol/L which can be safely and continuously used to human tissue and organs can be used to inhibit malignant tumors a sodium ion concentration in which is 451 mmol/L and pains caused by malignant tumors, and to suppress inflammation and edema a sodium ion concentration in which is 200-300 mmol/L and pains caused by the inflammation; in the specific implementation, the concentration of sodium ions outside the neuronal cell membrane in the human body can be increased from 150 to nearly 4524 mmol/L and then decreased back to 150 mmol/L; the solution is regularly and cyclically used every day to establish a repair and regeneration pattern that complies with the sodium ion spiral curve; the concentration of sodium ions in the solution starts from 150, reaches 4524, decreases to 150, and then reaches 4524 again, repeating in a regular cyclic pattern; when the concentration of sodium ions outside the neuronal cell membrane in the human body reaches or exceeds 440 mmol/L which is the concentration of sodium ions in the octopus cells, regenerative functions of cells is triggered; a cumulative regenerative frequency is maintained in a regular pattern every day, achieving an effect close to the regenerative capacity of an octopus.

4. The formula according to claim 1, wherein, the drug is administered noninvasively along a fixed pathway in the nervous system; accumulating number of times of the regeneration in a regular manner to suppress aging; the usage time and dosage are as follows: 1-2 times per day; for the eyes, the usage time is approximately one minute each time, and the dosage is 0.1-1 milliliters each time; for the nose, the usage time is approximately two minutes each time, and the dosage is 0.1-1 milliliters each time; for the mouth, the usage time is about 2 minutes each time, and the dosage is 5-10 milliliters each time; for the lung atomization, the usage time is about 30 minutes each time, and the dosage is 5-20 milliliters each time; for the reproductive organs, the usage time is about 10-20 minutes each time, and the dosage is 5-10 milliliters each time; for the nipples, the usage time is about 10 minutes each time, and the dosage is 10 milliliters each time; for the rectum, the usage time is about one minute each time, and the dosage is 5-10 milliliters each time; for the skin, the usage time is about 5-10 minutes each time, and the dosage is 60 milliliters each time; other methods, such as injection, involve minimal injection volumes of approximately 0.1 milliliters each time; for diseases which are more serious, the time for the non-invasive drug administration can be extended to several hours per day, with the nervous system taking in the drug actively and accelerating the inhibition of inflammation.

5. A preparation method for the formula according to claim 1, wherein, the formula is synthesized through physical methods.

6. An application of the formula according to claim 1 in the preparation for prevention, and/or treatment for the inflammation of the nervous system, the inflammation of the immune system and the degenerative disorder of the nervous system.

7. The application according to claim 6, the inflammation of the nervous system, the inflammation of the immune system and the degenerative disorder of the nervous system include: pain from cancers of any type and other inflammatory pains; cancers and inflammations associated with central nervous system and immune system; degenerative disorders of the central nervous system, such as Parkinson's disease, Alzheimer's disease, amyotrophic lateral sclerosis, depression, insomnia, impairments and declines of functions of the autonomic and peripheral nervous system, impairments and declines of eye function, retinal degeneration, impairments and declines of brain functions, impairments of memory functions, impairments and declines of functions of the spinal cord and spine, impairments and declines of the functions of internal organs such as the heart, lungs, kidneys, impairments and declines of muscle functions, impairments and declines of the function of teeth and gums, hair loss caused by scalp neuritis; impairments and declines of the intestinal motility and defecation function; impairments and declines of the urination function of the urinary system; impairments and declines of sexual function, impairments and declines of the function of the skin.

8. A drug for prevention and/or treatment of the inflammation of the nervous system, the inflammation of the immune system and the degenerative disorder of the nervous system, active ingredients of the drug include the formula as described in claims 1 and 2; a dosage form of the drug is any one of a spray, a liquid preparation, an injectable preparation, a tablet, a capsule, a granule, a suspension mixture and a pill.
